(19) United States
(12) Patent Application Publication
(10) Pub. No.: US 2026/0051411 A1
(43) Pub. Date: Feb. 19, 2026

(54) DATA ESTIMATION DEVICE, DATA ESTIMATION METHOD, AND RECORDING MEDIUM

(71) Applicant: NEC Corporation, Tokyo (JP)

(72) Inventors: Keisuke Suzuki, Tokyo (JP); Kentaro Nakahara, Tokyo (JP); Yuki Kosaka, Tokyo (JP); Kosuke Nishihara, Tokyo (JP); Fumiyuki Nihey, Tokyo (JP); Mana Hashimoto, Tokyo (JP)

(73) Assignee: NEC Corporation, Tokyo (JP)

(21) Appl. No.: 19/279,371

(22) Filed: Jul. 24, 2025

(30) Foreign Application Priority Data
Aug. 15, 2024 (JP) 2024-135754

(51) Int. Cl. G16H 50/70; G16H 10/60

(52) U.S. Cl. CPC G16H 50/70; G16H 10/60

(57) ABSTRACT

The data estimation device includes an acquisition unit, an estimation unit, and an output unit. The acquisition unit acquires health-related data of each person of different age groups. The estimation unit estimates the transition destination of the health-related data of the target person in a case where the age of the target person in the first age group becomes the second age group. The output unit outputs health-related data in a case where the age of the target person has increased based on the estimation result of the transition destination. With such a configuration, the data estimation device can support decision making related to a future health condition.

This application is based upon and claims the benefit of priority from Japanese Patent Application No. 2024-135754, filed on Aug. 15, 2024 the disclosure of which is incorporated herein in its entirety by reference.

### TECHNICAL FIELD

The present disclosure relates to a data estimation device and the like.

### BACKGROUND ART

The future health condition of a target person may be important in estimating assets necessary for receiving medical practice or the like in the future and grasping assets or the like that can be used for investment excluding assets necessary for receiving medical practice or the like. The prediction model construction device of JP 2016-173728 A clusters health condition data of a plurality of years for each of a plurality of subjects as a group of data for each subject and age group. The prediction model construction device of JP 2016-173728 A generates a prediction model that predicts the transition of the health condition based on a transition between clusters of data of the same subject.

### SUMMARY

An object of the present disclosure is to provide a data estimation device and the like that can easily estimate health-related data in the future.

A data estimation device according to one aspect of the present disclosure includes an acquisition means for acquiring health-related data of each person of different age groups, an estimation means for estimating a transition destination of health-related data of a target person in a case where an age of the target person in a first age group has become a second age group, and an output means for outputting the health-related data in a case where the age of the target person has increased based on an estimation result of the transition destination.

A data estimation method according to one aspect of the present disclosure includes acquiring health-related data of each person of different age groups, estimating a transition destination of health-related data of a target person in a case where an age of the target person in a first age group has become a second age group, and outputting the health-related data in a case where the age of the target person has increased based on an estimation result of the transition destination.

A non-transitory computer readable recording medium according to one aspect of the present disclosure records a data estimation program for causing a computer to execute a process of acquiring health-related data of each person of different age groups, a process of estimating a transition destination of health-related data of a target person in a case where an age of the target person in a first age group has become a second age group, and a process of outputting the health-related data in a case where the age of the target person has increased based on an estimation result of the transition destination.

### BRIEF DESCRIPTION OF THE DRAWINGS

FIG. 1 is a diagram illustrating an example of a configuration of a data estimation system in the present disclosure;

FIG. 2 is a diagram illustrating an example of data transition in the present disclosure;

FIG. 3 is a diagram illustrating an example of a configuration of a data estimation device in the present disclosure;

FIG. 4 is a diagram illustrating an example of a display screen of an estimation result of health-related data in the present disclosure;

FIG. 5 is a diagram illustrating an example of a display screen of an estimation result of health-related data in the present disclosure;

FIG. 6 is a diagram illustrating an example of a display screen of an estimation result of health-related data in the present disclosure;

FIG. 7 is a diagram illustrating an example of an operation flow of the data estimation device in the present disclosure;

FIG. 8 is a diagram illustrating an example of a configuration of a data estimation system in the present disclosure;

FIG. 9 is a diagram illustrating an example of a configuration of a data estimation device in the present disclosure;

FIG. 10 is a diagram illustrating an example of a display screen of an estimation result of a change value of health-related data in the present disclosure;

FIG. 11 is a diagram illustrating an example of a display screen of an estimation result of health-related data in the present disclosure;

FIG. 12 is a diagram illustrating an example of a display screen of a medical cost estimation result in the present disclosure;

FIG. 13 is a diagram illustrating an example of an operation flow of the data estimation device in the present disclosure;

FIG. 14 is a diagram illustrating an example of an operation flow of the data estimation device in the present disclosure; and

FIG. 15 is a diagram illustrating an example of a hardware configuration of the data estimation device in the present disclosure.

### EXAMPLE EMBODIMENT

### First Example Embodiment

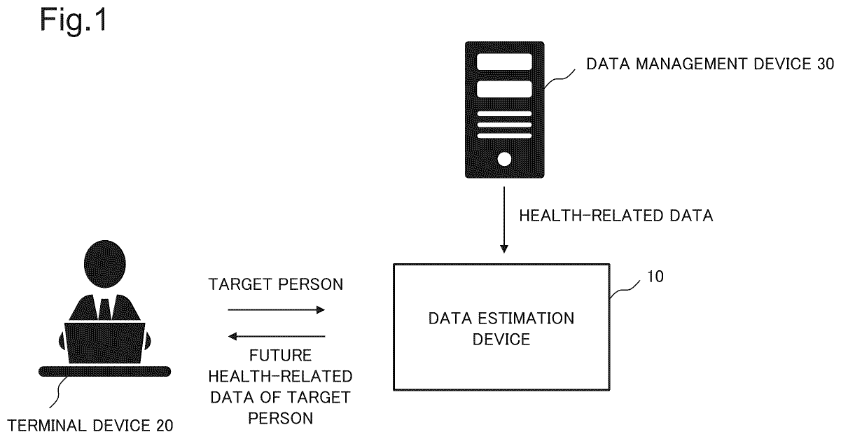

A first example embodiment of the present disclosure will be described in detail with reference to the drawings. FIG. 1 is a diagram illustrating an example of a configuration of a data estimation system. The data estimation system includes a data estimation device 10, a terminal device 20, and a data management device 30. The data estimation device 10 is connected to the terminal device 20 via, for example, a network. The data estimation device 10 is connected to the data management device 30 via, for example, a network. A plurality of terminal devices 20 and a plurality of data management devices 30 may be provided. The number of terminal devices 20 and the number of data management devices 30 can be appropriately set.

The data estimation system estimates, for example, health-related The data in a case where the age of the target person has increased. health-related data is, for example, a result of a medical examination. For example, the data estimation system estimates future health-related data of the target person based on the health-related data of the target person. For example, in a case where the current age of the target person is 50 years old, the data estimation system estimates health-related data when the target person becomes 70 years old based on the health-related data when the target person is 50 years old.

The target person is a person whose health-related data is to be estimated. The target person is, for example, a person who needs health-related data in planning for the future. Future plans are, for example, plans for asset management, housing or insurance. The future plan may be a plan related to exercise performed to maintain a healthy condition. The plan for the future is not limited to the above. The health-related data is, for example, data indicating health condition. A specific example of the health-related data will be described later.

The data estimation system estimates health-related data in a case where the age of the target person has increased using, for example, health-related data in each of a plurality of age groups. For example, it is assumed that a first age group and a second age group higher than the first age group are set. In this case, for example, the data estimation system estimates the health-related data of the target person when the age of the target person has increased from the first age group to the second age group based on the health-related data of each person belonging to the first age group and the health-related data of each person belonging to the second age group.

In a case where the health-related data is the result of the medical examination, the data estimation system estimates the result of the medical examination in a case where the age of the person who has undergone the medical examination has increased using, for example, the result of the medical examination for each age group performed in the current year. For example, the data estimation system estimates the health-related data of the target person in a case where the age of the target person to which the first age group belongs has increased to the second age group using the result of the medical examination on the person in the first age group and the result of the medical examination on the person in the second age group. The target person is a person whose health-related data in the future is to be estimated. The data estimation system estimates future health-related data of the target person using, for example, health-related data of the person different for each age group. Therefore, for example, the data estimation system estimates the future health-related data of the target person without requiring the data regarding the long-term health of the same person.

The result of the medical examination on the target person may be a result of a medical examination conducted in a year different from the year in which the medical examination in which the result of the medical examination for each age group was acquired was conducted. The result of the medical examination for each age group may be the result of the medical examination performed in a plurality of years. In this case, when the result of the medical examination on the same person is included in the results of the medical examinations in different years, the data estimation system regards, for example, each of the results of the medical examinations on the same person included in the results of the medical examinations in different years as the result of the medical examinations on different persons, and performs estimation.

The data estimation system may classify health-related data of each age group into data groups, and estimate a transition of the health-related data in a case where the age of the target person has increased as a transition between the data groups. For example, in a case where the age of the person whose result of the medical examination is classified into a first data group in the first age group becomes the second age group, the data estimation system estimates which data group of the second age group the result of the medical examination classified into the first data group belongs to. Then, for example, the data estimation system estimates a result of the medical examination in a case where the age of the target person has increased from the first age group to the second age group based on the result of the medical examination of the target person.

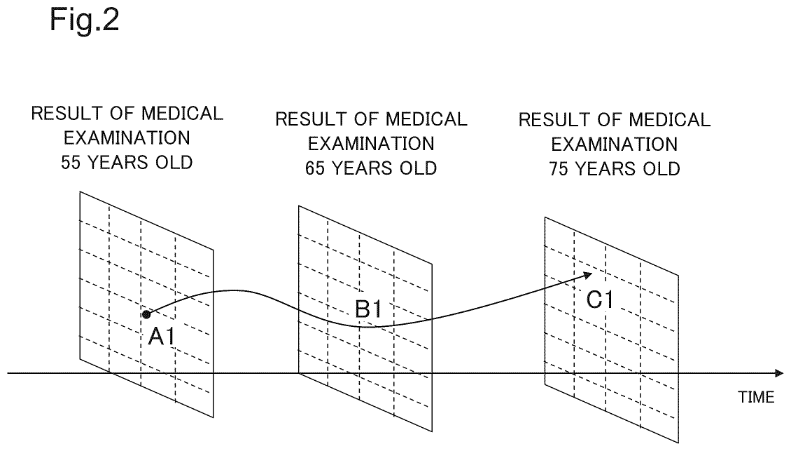

FIG. 2 is a diagram schematically illustrating an example in which data transitions when age has increased in a case where health-related data is a result of a medical examination. In the example of FIG. 2, for example, 55 years of age is relevant to the first age group, and 75 years of age is relevant to the second age group. In the example of FIG. 2, the result of the medical examination on the person belonging to the data group A1 at the age of 55 indicates that the person transitions to the data group C1 via the result of the medical examination at the age of 65 in a case where the age of the person has increased to the age of 75. In the example of FIG. 2, the result of the medical examination on the person belonging to the data group A1 at the age of 55 transitions to the data group B1 in the result of the medical examination at the age of 65. That is, in the example of FIG. 2, the data of the data group A1 transitions to the data group C1 via the data group B1 between different age groups. In this case, when the result of the medical examination on the target person at the age of 55 is included in the data group A1, the data estimation system estimates that the result of the medical examination that the target person receives when the subject turns 75 is within the range of the data group C1.

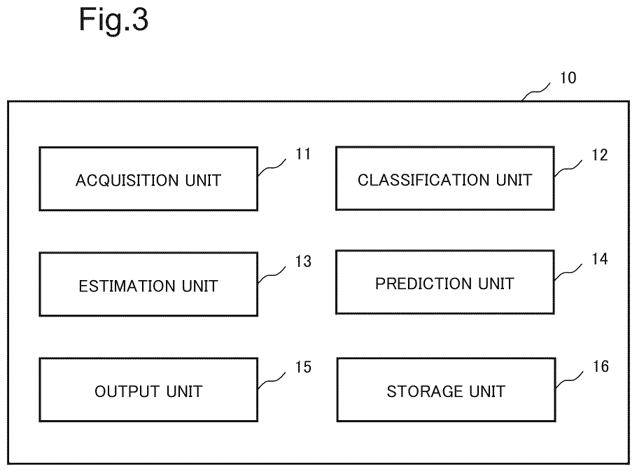

Here, an example of a configuration of the data estimation device will be described. FIG. 3 is a diagram illustrating an example of a configuration of the data estimation device 10. The data estimation device 10 includes an acquisition unit 11, an estimation unit 13, and an output unit 15 as a basic configuration. The data estimation device 10 further includes, for example, a classification unit 12, a prediction unit 14, and a storage unit 16.

The acquisition unit 11 acquires health-related data of each person of different age groups. The health-related data may also include an index calculated from the health-related data. For example, the acquisition unit 11 acquires the health-related data in a state in which the information indicating the age of the person relevant to each piece of the health-related data is associated. For example, the acquisition unit 11 may acquire health-related data in a state in which information indicating an attribute of a person relevant to each piece of health-related data is associated. The attribute is, for example, information indicating a group in which a difference in tendency of health-related data may occur due to a difference in the attribute. For example, the tendency of health-related data may be different between a person who lives in a cold region and a person who lives in a warm region. In such a case, for example, information indicating a place of residence is used as the attribute. The attribute is, for example, information of one or more items of gender, domicile, nationality, occupation, previous disease, and previous disease of a family. The attribute is not limited to the above. For example, the acquisition unit 11 acquires, from the data management device 30, health-related data of each person of different age groups.

The health-related data is, for example, data indicating a health condition. The health-related data is, for example, data of one or more items among a result of a medical examination, a test value in a hospital, vital data, presence or absence of onset of a disease, a probability of onset of a disease, opinions of a physician, an exercise function, necessity of care, and a degree of necessary care. The result of the medical examination is, for example, data of one or more items among a height, a weight, a visual acuity, a blood pressure, an abdominal circumference, a tension, a measured value in a blood test, an image diagnosis result, and a physician interview result measured in the medical examination. The health-related data may also include costs required for maintaining the health condition or daily life. The health-related data is not limited to the above.

In a case where the health-related data is the result of the medical examination, the acquisition unit 11 acquires, for example, the result of the medical examination within a predetermined period as the health-related data. The predetermined period is, for example, a period in which a sufficient number of pieces of data can be collected, and is set as a period in which the tendency of the data does not change. The fact that the tendency of data does not change means that, for example, data can be acquired with the same standard without changing the standard of implementation of a medical examination. The predetermined period is, for example, within the same year. The predetermined period may be a plurality of years. The predetermined period may be one month or multiple months. The predetermined period is not limited to the above. In a case where the health-related data is a result of a medical examination, the acquisition unit 11 may acquire, for example, a result of a medical examination in a predetermined group as health-related data.

The predetermined group is, for example, a group in which the health condition of persons belonging to the group tends to be different from that of other groups. The predetermined group is, for example, a region, a company, an industry type, or a job type. The predetermined group is not limited to the above. For example, it is assumed that there is a difference in tendency to develop a disease between a person engaged in the fishing industry and a person engaged in another industry type. In this case, in a case where the health-related data of persons engaged in the fishing industry is estimated, the predetermined group is set as, for example, a group of persons engaged in the fishing industry. In this case, for example, the result of the medical examination performed in the fishery cooperative is used as health-related data.

The acquisition unit 11 acquires, for example, health-related data of the target person. The acquisition unit 11 acquires, for example, the latest health-related data among the health-related data of the target person. For example, in a case where the health-related data is a result of a medical examination, the acquisition unit 11 acquires a result of a medical examination that the target person has received most recently. The acquisition unit 11 acquires, for example, the age of the target person. The age of the target person may be, for example, the age at the time when the health-related data of the target person is measured. The acquisition unit 11 may acquire the attribute of the target person. The acquisition unit 11 acquires, for example, information for specifying the target person from the terminal device 20. The acquisition unit 11 acquires health-related data from the data management device 30, for example. The acquisition unit 11 may acquire health-related data of the target person from the terminal device 20.

In a case where health-related data of each age group is classified into a data group and a transition destination in a case where the age has increased is estimated, the classification unit 12 classifies the health-related data in each age group into a data group, for example. For example, the classification unit 12 generates a probability density distribution of the health-related data for each age group based on the classified data group. In a case of generating a probability density distribution of the health-related data, for example, the classification unit 12 classifies the health-related data into a data group for each predetermined section for each age group. Then, for example, the classification unit 12 generates a probability density distribution of health-related data for each age group by using a data group classified for each predetermined section. For example, in a case where two items of data among the health-related data are used, the classification unit 12 generates a probability density distribution of the health-related data for each age group by classifying the health-related data on a grid having each of the two items of data as an axis. In this case, the data group is, for example, data classified into each of grids.

In a case where the health-related data is blood pressure, the classification unit 12 classifies each of the systolic blood pressure and the diastolic blood pressure into a data group of every 5 mmHg for each age group, for example. In a case where the health-related data is blood pressure, the classification unit 12 may classify the data into a two-dimensional data group in which the systolic blood pressure and the diastolic blood pressure are associated with each other. For example, the classification unit 12 classifies the health-related data into any of grids obtained by dividing each of the systolic blood pressure and the diastolic blood pressure in each age group by 5 mmHg. Then, the classification unit 12 generates a probability density distribution of blood pressure data for each age group.

For example, in a case where the health-related data is HbAlc and glucose, the classification unit 12 classifies the health-related data into any of grids obtained by dividing HbAlc by 0.1 percent and glucose by 5 mg/dL in each age group.

For example, the classification unit 12 generates a probability density distribution of health-related data based on a data group obtained by classifying health-related data. For example, the classification unit 12 generates a probability density distribution of health-related data by dividing the number of pieces of data of each data group by the total number of pieces of data.

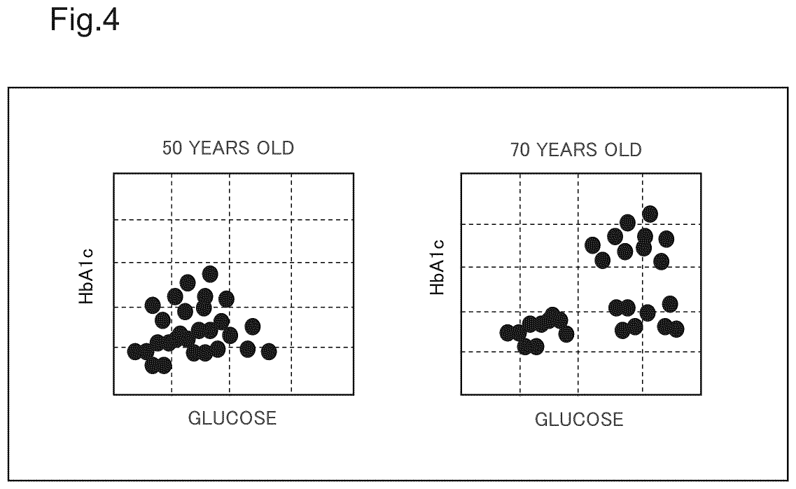

FIG. 4 is an example of health-related data at the age of 50 and at the age of 70. An example of the health-related data in FIG. 4 is two-dimensional data having glucose and HbAlc test data as axes. In the health example of FIG. 4, many people have small values of both glucose and HbAlc at the age of 50, whereas there are a group in which the value of glucose is increased, a group in which the values of both glucose and: HbAlc are increased, and a group in which the change in the value is small at the age of 70.

The classification unit 12 may classify the health-related data based on the attribute of the person relevant to each piece of the health-related data. For example, in a case where the attribute is gender, the classification unit 12 classifies health-related data into female data and male data.

The classification unit 12 may classify health-related data based on the attribute of the target person. For example, the classification unit 12 classifies health-related data of a person having the same attribute as that of the target person among the health-related data acquired by the acquisition unit 11. In a case where the health-related data is classified into the data group and the health-related data in a case where the age of the target person has increased is estimated, the classification unit 12 may classify the health-related data into the data group based on the attribute of the target person.

In a case where the disease onset probability is predicted, the classification unit 12 may generate a probability density distribution of the disease onset probability. For example, the classification unit 12 generates a probability density distribution of the disease onset probability for each age group based on the disease onset probability predicted by the prediction unit 14.

In a case where the health-related data acquired by the acquisition unit 11 is not classified into data for each age group, the classification unit 12 classifies the health-related data into data for each age group based on the age category estimated by the estimation unit 13. For example, in a case where the estimation unit 13 estimates health-related data in categories for every 10 years of age, the classification unit 12 classifies the health-related data into data for every 10 years of age. In a case where the health-related data is classified into a data group and the health-related data in a case where the age of the target person has increased is estimated, the classification unit 12 may classify the health-related data into data for each age group based on the age category estimated by the estimation unit 13, and generate the data of the probability density distribution for each age group. For example, the classification unit 12 generates data of probability density distribution for every 10 years old by using health-related data classified for every 10 years old.

The estimation unit 13 estimates the transition destination of the health-related data of the target person in a case where the age of the target person in the first age group becomes the second age group. For example, the estimation unit 13 estimates a transition destination of health-related data of the target person using an algorithm related to the optimal transportation problem. The algorithm related to the optimal transportation problem is, for example, an algorithm for performing optimization processing by optimal transport. In a case where health-related data of each age group is classified into a data group, and a transition destination in a case where the age has increased is estimated, the estimation unit 13 estimates a second data group to be a transition destination of the health-related data of the target person in a case where the age of the target person in the first age group of which the health-related data is classified into the first data group becomes the second age group. For example, the estimation unit 13 estimates a transition destination in a case where transition is made from the first data group on the probability density distribution of health-related data in the first age group to the data group on the probability density distribution of health-related data in the second age group as a second data group.

For example, the estimation unit 13 calculates a state transition probability in a case where transition is made from each piece of health-related data in the first age group to each piece of health-related data in the second age group. The estimation unit 13 calculates the state transition probability from each piece of data in the first age group to each piece of data in the second age group using, for example, an algorithm of the optimal transportation problem.

The estimation unit 13 calculates a state transition probability in a case where transition is made from each piece of data on the probability density distribution in the first age group to each piece of health-related data in the second age group, for example, using an algorithm of the optimal transportation problem. For example, the estimation unit 13 calculates the state transition probability by estimating π(dx, dy) at which the transportation cost c (x, y) is minimized in the following Expression 1.

$$\begin{matrix}{\int }_{x \times y} c ( x , y ) π ( dxdy ) , & π ∈ \prod ( μ , v ) .\end{matrix} \qquad [ Math . 1 ]$$

In Expression 1 above, x and y are health-related data. In Expression 1 above, u and v are probability density distributions relevant to the first age group and the second age group. The estimation unit 13 calculates the state transition probability at which the transportation cost is minimized using, for example, the least square error of the cost function.

For example, the estimation unit 13 estimates the transition destination of the health-related data of the target person in a case where the age of the target person in the first age group becomes the second age group based on the calculated state transition probability. For example, the estimation unit 13 estimates, as the calculated expected value of the state transition probability, data to be the transition destination in a case where the age of the target person becomes the second age group. Then, the estimation unit 13 estimates, for example, the estimated transition destination data as health-related data in a case where the age of the target person is in the second age group.

In a case where health-related data of each age group is classified into a data group, and a transition destination in a case where the age has increased is estimated, the estimation unit 13 estimates a data group to be a transition destination of the health-related data of the target person in a case where the age of the target person in the first age group of which the health-related data is classified into the first data group becomes the second age group based on the calculated state transition probability, for example. For example, the estimation unit 13 estimates a data group having the highest calculated state transition probability as a data group to be a transition destination in a case where the age of the target person becomes the second age group. Then, the estimation unit 13 estimates, for example, data indicated by the estimated data group of the transition destination as health-related data in a case where the age of the target person becomes the second age group. For example, the estimation unit 13 estimates a mode of data indicated by the estimated data group of the transition destination as health-related data in a case where the age of the target person becomes the second age group. The estimation unit 13 may estimate the minimum value or the maximum value of the data indicated by the estimated data group of the transition destination as health-related data in a case where the age of the target person becomes the second age group.

For example, the estimation unit 13 calculates a state transition probability in a case where transition is made from each piece of health-related data in the first age group to each piece of health-related data in the second age group for each piece of data of a predetermined item among pieces of health-related data. For example, the estimation unit 13 calculates the state transition probability by changing the setting values of the first age group and the second age group for each piece of data of the predetermined item. That is, the estimation unit 13 calculates the state transition probability for each combination of the predetermined item, the first age group, and the second age group, for example. Then, the estimation unit 13 stores, for example, the state transition probability for each combination of the predetermined item, the first age group, and the second age group in the storage unit 16 as a database. The estimation unit 13 stores, for example, the state transition probability optimized for each combination of the predetermined item, the first age group, and the second age group as a database, thereby estimating future health-related data of the target person.

The data of the predetermined item is, for example, data of an item related to a predetermined disease. The predetermined disease is, for example, a disease that greatly affects the life of the target person when affected. The predetermined disease may be a disease that affects a large number of people with increasing age. The predetermined disease is, for example, cancer, myocardial infarction, diabetes, hypertension, and brain disease. The predetermined disease is not limited to the above. The predetermined item is not limited to the above.

For example, the estimation unit 13 selects the data of the state transition probability from the database based on the age of the target person and the item to be estimated. Then, the estimation unit 13 estimates, for example, health-related data in a case where the age of the target person becomes the second age group based on the data of the selected state transition probability.

For example, the estimation unit 13 estimates a second data group to be a transition destination from the first data group using an item related to a predetermined disease among health-related data as a variable.

For example, in a case where the health-related data is the result of the medical examination, when the predetermined disease is diabetes, the second data group to be the transition destination from the first data group may be estimated using the measured values of HbAlc and glucose used as indexes for diagnosing diabetes as variables.

The estimation unit 13 may estimate a transition destination of the data group among a plurality of age groups. For example, the estimation unit 13 estimates a second data group to be a transition destination from the first data group via a data group of health-related data in at least one or more age groups between the first age group and the second age group. For example, in a case where the first age group is set as 50's and the second age group is set as 70's, the estimation unit 13 estimates a data group to be the transition destination via a data group of health-related data in 60's. There may be more than one age group between the first age group and the second age group. The interval between the age groups may be narrower than 10 years old.

The estimation unit 13 may estimate the transition destination of the data of the disease onset probability in the target person in a case where the age of the target person becomes the second age group based on the disease onset probability in the first age group. For example, the estimation unit 13 calculates a state transition probability between the disease onset probability in the first age group and the disease onset probability in the second age group. Then, for example, the estimation unit 13 estimates the transition destination of the data of the probability of the disease in the target person in a case where the age of the target person becomes the second age group based on the calculated state transition probability.

For example, the prediction unit 14 predicts the disease onset probability in a case where a person of the first age group becomes the second age group based on data of the second data group estimated as a transition destination from the first data group.

For example, the prediction unit 14 predicts the disease onset probability in a case where a person in the first age group becomes the second age group using a disease prediction model based on data estimated as a transition destination of health-related data of the target person. The disease prediction model is, for example, a machine learning model that predicts the disease onset probability of a target person using health-related data of the target person as an input. The disease prediction model is generated, for example, by performing machine learning on a relationship between health-related data of each of a plurality of persons and presence or absence of onset of a disease in each of the persons. The disease prediction model is generated by deep learning using a neural network, for example. The learning algorithm for generating the disease prediction model is not limited to the above. The disease prediction model is generated, for example, in a system outside the data estimation device 10. The disease prediction model may be generated by, for example, learning means (not illustrated) inside the data estimation device 10.

The prediction unit 14 may predict the disease onset probability for each age group in each data group into which health-related data is classified. For example, the prediction unit 14 predicts the disease onset probability in each data group based on health-related data of each age group.

The prediction unit 14 may predict the medical cost of the target person. The medical cost is, for example, a cost for the target person to receive medical care. Medical care may include caregiving. The prediction unit 14 predicts the medical cost of the target person based on the disease onset probability. The relationship between the disease onset probability and the medical cost is set as, for example, tabular data. The prediction unit 14 may predict the medical cost of the target person using a function having the predicted value of the medical cost as an objective variable and the disease onset probability as an explanatory variable. The function used for prediction of the medical cost is set, for example, for each disease.

The output unit 15 outputs health-related data in a case where the age of the target person has increased based on the estimation result of the transition destination. The output unit 15 outputs, for example, health-related data of the target person in the first age group and health-related data in a case where the age of the target person has increased to the second age group. For example, in a case where the first age group is 50 years old and the second age group is 70 years old, the output unit 15 outputs health-related data of the target person at 50 years old and an estimation result of the health-related data at 70 years old.

The output unit 15 may output an estimation result of health-related data relevant to each predetermined disease. The output unit 15 may output the disease onset probability in a case where the age of the target person in the first age group becomes the second age group as the health-related data. The output unit 15 may output a sentence describing the estimation result of the health-related data of the target person. The relationship between the estimation result of the health-related data and the sentence describing the estimation result is set as, for example, tabular data. The output unit 15 outputs, for example, health-related data in a case where the age of the target person has increased to the terminal device 20.

The output unit 15 may output a prediction result of the medical cost necessary for the target person to receive medical care in the future as an estimation result of health-related data in a case where the age of the target person has increased. The output unit 15 may output at least one of health-related data and a prediction result of medical cost in a case where the age of the target person has increased as time series data. The time series data is, for example, a graph in which a horizontal axis is time and a vertical axis is health-related data or a prediction result of medical cost in a case where the age of the target person has increased.

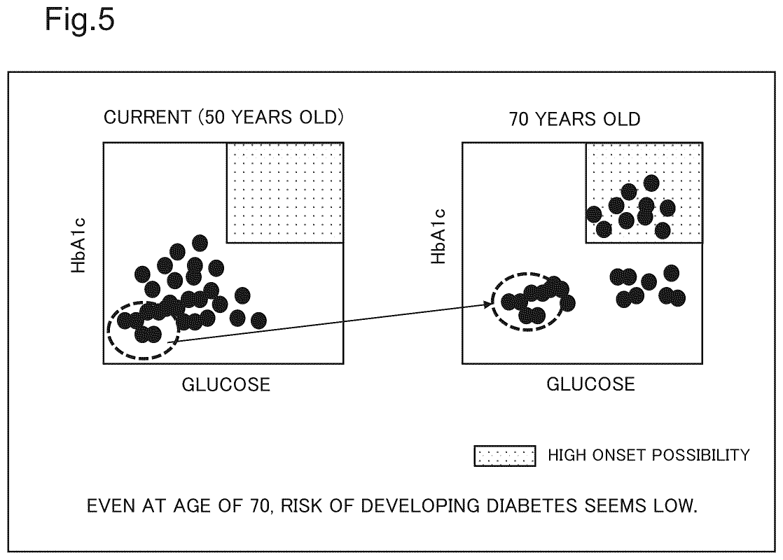

FIG. 5 is an example of a display screen that displays an estimation result of future health-related data of the target person. The example of the display screen in FIG. 5 is, for example, a display screen showing an estimation result of health-related data when the age of the target person has increased from 50 years old to 70 years old in a case where the first age group is 50 years old and the second age group is 70 years old. The example of the display screen in FIG. 5 indicates that the health-related data of the person in which the health-related data at the age of 50 is present inside the dashed line transitions into the dashed line of the data at the age of 70 in a case where the age has increased to the age of 70. In the example of the display screen of FIG. 5, even in a case where the age has increased to 70 years old, since data exists in a region where the onset probability is low, a sentence “even at the age of 70, the risk of developing diabetes seems low” is displayed.

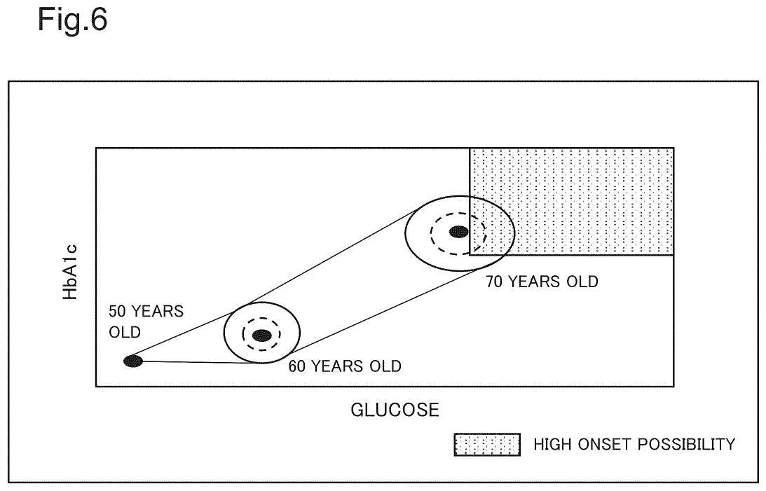

FIG. 6 is an example of a display screen that displays an estimation result of future health-related data of the target person together with an error range of the estimated value. In the example of the display screen of FIG. 6, the estimated values of HbAlc and glucose in a case where a 50 year old person becomes 60 years old and 70 years old are displayed. In the example of the display screen in FIG. 6, the error range of the estimated value at the age of 60 and the error range of the estimated value at the age of 70 are displayed using concentric circles. In the example of the display screen in FIG. 6, the error range of the estimated value at the age of 60 and the error range of the estimated value at the age of 70 are displayed using two concentric circles of a broken line and a solid line. The output unit 15 outputs an error range of the estimated value based on, for example, the state transition probability. For example, in a case where two stages of concentric circles are output, the output unit 15 outputs the error range of the estimated value based on the criterion of the state transition probability set in two stages.

The storage unit 16 stores, for example, information regarding processing of estimating future health-related data of the target person.

The storage unit 16 stores, for example, a state transition probability for each combination of a predetermined item, the first age group, and the second age group as a database. The storage unit 16 stores, for example, health-related data of the target person. The storage unit 16 stores, for example, an estimation result of health-related data of the target person.

The storage unit 16 stores a disease prediction model. The disease prediction model may be stored in storage means outside the data estimation device 10.

The terminal device 20 is, for example, a terminal device that accesses the data estimation device 10 and is used for a process of estimating future health-related data. The terminal device 20 acquires, for example, an estimation result of future health-related data of the target person from the output unit 15 of the data estimation device 10. Then, the terminal device 20 outputs an estimation result of future health-related data of the target person to a display device (not illustrated), for example.

The terminal device 20 is used by, for example, a person or a target person who gives advice on health or assets to the target person. A person who advises the target person is, for example, a medical worker, an insurance person, a human resource person, a financial planner, or a person in charge of a financial institution. The medical worker is a physician, nurse, physical therapist, pharmacist, laboratory technician, or even a consultant. The medical worker is not limited to the above. The person who gives advice to the target person is not limited to the above.

As the terminal device 20, for example, a personal computer, a tablet computer, a smartphone, or a smartwatch can be used. The information processing device used for the terminal device 20 is not limited to the above.

The data management device 30 stores, for example, health-related data. For example, the data management device 30 stores health-related data in association with a measurement date of the health-related data and the age of the person relevant to the health-related data. The data management device 30 may store the attribute of the person relevant to the health-related data in association with the health-related data. The data management device 30 outputs health-related data to the acquisition unit 11 of the data estimation device 10, for example.

In a case where the health-related data is the result of the medical examination, the data management device 30 stores, for example, the execution date of the medical examination, the age of the person who has received the medical examination, and the result of the medical examination in association with each other. The attribute is, for example, information of one or more items of gender, domicile, nationality, occupation, previous disease, and previous disease of a family. The attribute is not limited to the above. The execution date of the medical examination may be information indicated by the month, year, or year in which the medical examination was conducted. The data management device 30 may store the result of the medical examination as a database classified based on at least one of the execution date of the medical examination and the attribute of the person who has received the medical examination.

For example, the data management device 30 may store the result of the medical examination as anonymized information or pseudonymized information. The anonymized information is, for example, information processed so that an individual cannot be identified even if the anonymized information is collated with other information. The pseudonymized information is, for example, information processed so that an individual cannot be specified by itself but can be specified by collating with other information.

The data management device 30 stores, for example, a result of a medical examination performed in a predetermined group. The predetermined group is, for example, a group that performs a medical examination. The predetermined group is, for example, a municipality, a company, an association, a cooperative association, a school, or a health insurance association. The predetermined group is not limited to the above. The data management device 30 may store results of medical examinations of a plurality of groups.

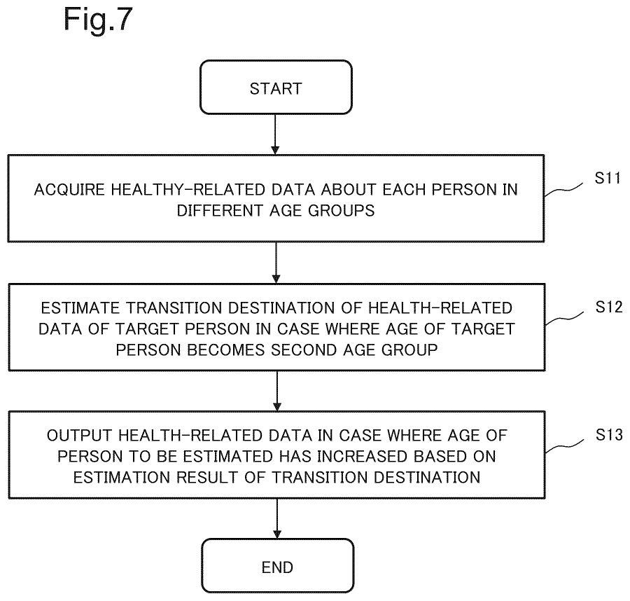

Processing in which the data estimation device 10 estimates health-related data in a case where the age of the target person has increased will be described. FIG. 7 illustrates an example of an operation flow for the processing in which the data estimation device 10 estimates health-related data in a case where the age of the target person has increased.

The acquisition unit 11 acquires health-related data of each person of different age groups (step S11). The acquisition unit 11 acquires health-related data from the data management device 30, for example. In a case where the health-related data is acquired, the estimation unit 13 estimates the transition destination of the health-related data of the target person when the age of the target person in the first age group becomes the second age group (step S12).

When the transition destination of the data is estimated, the output unit 15 outputs health-related data in a case where the age of the target person has increased based on the estimation result of the transition destination (step S13). The output unit 15 outputs, for example, health-related data in a case where the age of the target person has increased to the terminal device 20.

Each processing in the data estimation device 10 may be executed in a distributed manner in a plurality of information processing devices connected via a network. For example, the processing in the classification unit 12 and the estimation unit 13 and the processing in the prediction unit 14 may be performed in another information processing device. Which information processing device performs each process in the data estimation device 10 can be appropriately set.

The data estimation device 10 estimates the transition destination of the health-related data of the target person in a case where the age of the target person in the first age group becomes the second age group. Then, the data estimation device 10 outputs health-related data in a case where the age of the target person has increased based on the estimation result of the transition destination. By estimating the health-related data in a case where the age of the target person has increased in this manner, the data estimation device 10 can easily estimate the health-related data in a case where the age of the target person has increased.

The data estimation device 10 can estimate the future health-related data without requiring the long-term health-related data of the same person by estimating the health-related data in a case where the age of the target person has increased as described above. Therefore, the data estimation device 10 can easily estimate health-related data in a case where the age has increased. By estimating health-related data in a case where the age has increased, the data estimation device 10 can support, for example, decision making regarding health performed by the target person.

By predicting the disease onset probability, the data estimation device 10 can provide information regarding the future disease onset risk of the target person. By estimating the medical cost necessary for taking medical care in the future, the data estimation device 10 can provide information for creating the asset planning accuracy of the target person.

### Second Example Embodiment

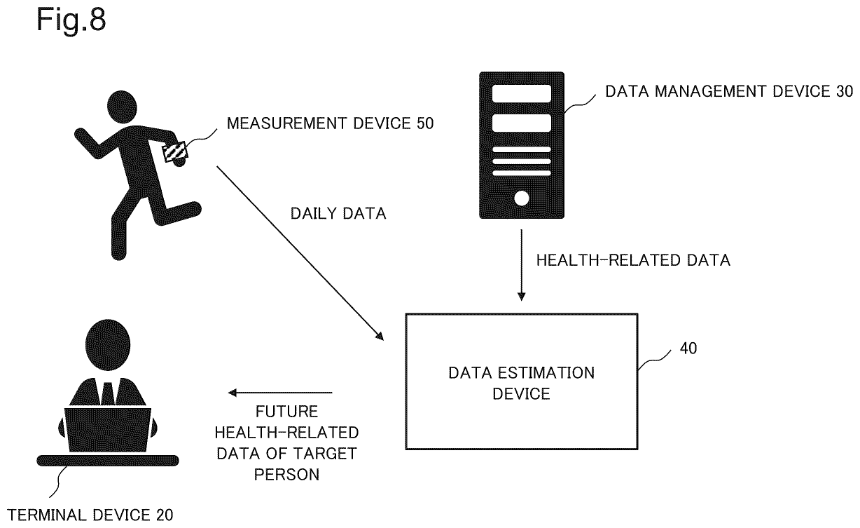

A second example embodiment of the present disclosure will be described in detail with reference to the drawings. FIG. 8 is a diagram illustrating an example of a configuration of a data estimation system. The data estimation system includes a data estimation device 40, a terminal device 20, a measurement device 50, and a data management device 30. The data estimation device 40 is connected to the terminal device 20 via, for example, a network. The data estimation device 40 is connected to the measurement device 50 via, for example, a network. The data estimation device 40 is connected to the data management device via, for example, a network. A plurality of terminal devices 20, measurement devices 50, and data management devices 30 may be provided. The number of terminal devices 20, the number of measurement devices 50, and the number of data management devices 30 can be appropriately set.

The data estimation system of the present example embodiment estimates future health-related data of the target person based on, for example, health-related data of the target person and daily data. The daily data is, for example, data acquired in daily life of the target person. The data acquired in daily life of the target person is data related to at least one of a behavior in daily life and a physical condition in daily life.

The behavior in daily life is, for example, a behavior that can affect the health condition among the behaviors of the target person. Health conditions may include cognitive function. The daily data of the target person is data of one or more behaviors among vital data, the number of walks, a walking distance, a running distance, a sleeping time, a meal amount, a meal content, a content of a performed sport, a content of muscle strength training, a performance time of muscle strength training, a performance time of sports, a performance time of yoga, and a reading amount.

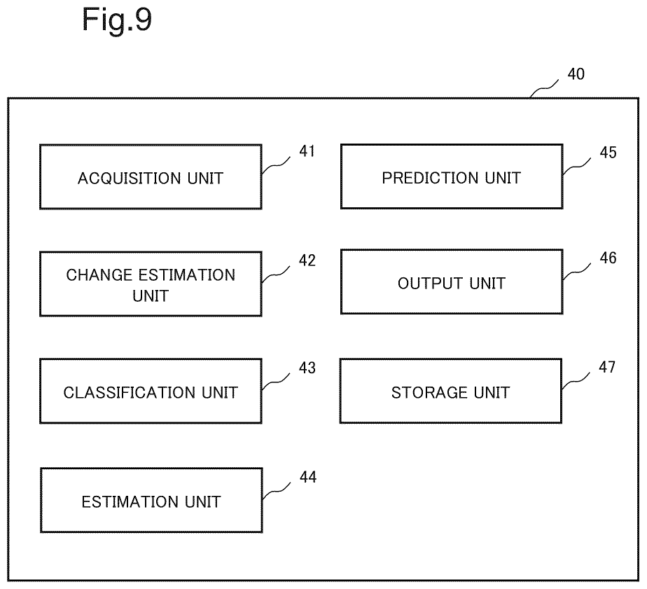

The daily data is measured using, for example, the measurement device 50. The daily data may be data input by the target person or other persons. The other persons are, for example, a family member, an instructor, a medical worker, a caregiver, or a care supporter. The medical worker is a physician, nurse, physical therapist, pharmacist, laboratory technician, or even a consultant. The medical worker is not limited to the above. The other persons are not limited to the above. Here, a specific example of the configuration of the data estimation device 40 will be described. FIG. 9 is a diagram illustrating an example of a configuration of the data estimation device 40. The data estimation device 40 includes, for example, an acquisition unit 41, a change estimation unit 42, a classification unit 43, an estimation unit 44, a prediction unit 45, an output unit 46, and a storage unit 47.

The acquisition unit 41 has a function similar to that of the acquisition unit 11 of the first example embodiment. The acquisition unit 41 further acquires, for example, daily data of the target person. The acquisition unit 41 acquires, for example, daily data of the target person after a time point at which the health-related data of the target person is measured. For example, in a case where the health-related data is the result of the medical examination, the acquisition unit 41 acquires daily data after the day on which the target person has undergone the medical examination. In a case where the health-related data is the result of the medical examination, the acquisition unit 41 may acquire daily data after the target person receives an explanation of the result of the medical examination from the medical worker.

The acquisition unit 41 may further acquire daily data of the target person before a time point at which the health-related data of the target person is measured. The daily data of the target person before the time point at which the health-related data of the target person is measured is used, for example, to calculate a change amount of the daily data before and after the time point at which the health-related data of the target person is measured.

The acquisition unit 41 may further acquire information indicating the content of a behavior performed to improve the health condition and the target value. For example, in a case where the behavior for improving the health condition is walking, the behavior content related to health improvement is information indicating that the behavior for improving the health condition is walking and a target value of the number of steps. The target value of the number of steps is, for example, the number of steps achieved per day. The target value of the number of steps is not limited to the number of steps per day. The target value may be indicated by a distance. The acquisition unit 41 acquires, for example, daily data of the target person from the measurement device 50. The acquisition unit 41 may acquire the daily data via the terminal device 20.

For example, the change estimation unit 42 estimates a change in health-related data caused by the daily life of the target person based on the daily data of the target person. The change estimation unit 42 estimates, for example, health-related data after a change has occurred due to daily life of the target person. The change estimation unit 42 may estimate a change amount of health-related data due to a change caused by daily life of the target person.

For example, in a case where the target person continuously runs, the test values of cholesterol, neutral fat, blood glucose level, and Hbalc among the blood test data of the target person in the medical examination can be improved. In such a case, the change estimation unit 42 estimates the data of the blood test reflecting the effect of the running based on, for example, the running distance of the target person per day and the measured value of the blood test data.

The change estimation unit 42 may estimate the health-related data based on the change amount of the daily data before and after the time point the health-related data of the target person is measured. The health-related data may be estimated based on the change amount of the daily data in a case where the target person changes the daily life. For example, the change estimation unit 42 estimates health-related data after a change has occurred due to daily life using a change estimation model. The change estimation model is, for example, a machine learning model that estimates health-related data after a change has occurred due to daily life using daily data of the target person as an input. The change estimation model is generated, for example, by machine-learning a relationship between health-related data and daily data of each of a plurality of persons and health-related data after a change has occurred due to daily life in each person. The change estimation model is generated by deep learning using a neural network, for example. The learning algorithm for generating the change estimation model is not limited to the above. The change estimation model is generated, for example, in a system outside the data estimation device 10. The change estimation model may be generated by, for example, a learning means (not illustrated) inside the data estimation device 10.

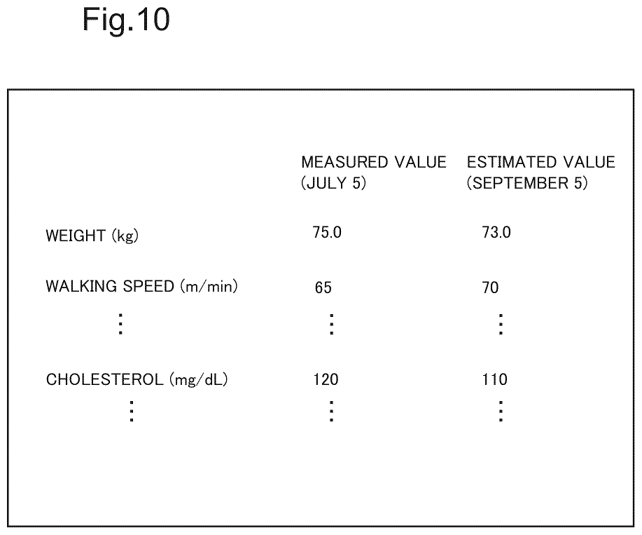

FIG. 10 illustrates an example of an estimation result of health-related data after a change has occurred due to daily life of the target person. In the example of the estimation result in FIG. 10, the measured value is health-related data actually measured. For example, in a case where the health-related data is the result of a blood test in a medical examination, the measured value is a value obtained by measuring blood in the blood test performed in the medical examination. In the example of the estimation result in FIG. 10, the estimated value indicates a value of health-related data after a change has occurred due to daily life. That is, the estimated value is, for example, an estimated value of health-related data estimated based on daily data. For example, the change estimation unit 42 calculates a value of health-related data after a change has occurred due to daily life as an estimated value illustrated in the example of the estimation result of FIG. 10 based on the daily data of the target person.

The change estimation unit 42 may estimate health-related data in a case where a behavior for improving the health condition is performed as desired based on the content of the behavior for improving the health condition and the information indicating the target value. For example, the change estimation unit 42 may estimate health-related data in a case where a behavior for improving health condition is performed as desired using a change estimation model. In this case, for example, the change estimation unit 42 inputs the target value as daily data to the change estimation model, thereby estimating health-related data in a case where a behavior for improving the health condition is performed as desired. The change estimation unit 42 may estimate health-related data in a case where a behavior for improving health condition is performed as desired with reference to data in a table format indicating a relationship between a content and a target value of a behavior for improving health condition and a change amount of health-related data.

The classification unit 43 has a function similar to that of the classification unit 12 of the first example embodiment. The classification unit 43 may classify health-related data after a change has occurred due to daily life estimated by the change estimation unit 42 into a data group. For example, the classification unit 43 generates a probability density distribution of health-related data for each age group based on a data group obtained by classifying health-related data after a change has occurred due to daily life.

The estimation unit 44 has a function similar to that of the estimation unit 13 of the first example embodiment. For example, the estimation unit 44 further calculates a state transition probability in a case where transition is made from each piece of health-related data in the first age group, which is a measured value, to each piece of health-related data in the second age group. Then, for example, the estimation unit 44 estimates a transition destination of the health-related data of the target person in a case where the age of the target person becomes the second age group using the health-related data estimated by the change estimation unit 42 as data in the first age group of the target person. Then, the estimation unit 44 estimates the data of the transition destination as health-related data in a case where the age of the target person has increased to the second age group.

For example, the estimation unit 44 stores the state transition probability for each combination of the predetermined item, the first age group, and the second age group in the storage unit 47 as a database. The predetermined item is the same as the predetermined item of the first example embodiment. For example, the estimation unit 44 selects the data of the state transition probability from the database based on the age of the target person and the item to be estimated. Then, for example, the estimation unit 44 estimates health-related data in a case where the age of the target person becomes the second age group using the health-related data estimated by the change estimation unit 42 as data in the first age group of the target person based on the data of the selected state transition probability.

For example, it is assumed that the first age group of the target person is 50 years old and the second age group is 70 years old. It is assumed that the health-related data is the result of the medical examination. In this case, for example, the change estimation unit 42 estimates a change in the result of the medical examination taken at the age of 50 based on the daily data of the target person who is 50 years old. For example, the change estimation unit 42 estimates a result of the medical examination in a case where the target person takes the medical examination after the target person changes according to the daily life of the target person, based on the data of the state transition probability in a case where the age is increased from 50 to 70 years old. Then, the estimation unit 44 estimates the result of the medical examination in a case where the target person takes the medical examination when the target person becomes 70 years old using the estimated result of the medical examination as the result of the medical examination of the target person at the age of 50.

The estimation unit 44 may calculate the state transition probability in a case where transition is made from each piece of health-related data in the first age group estimated from the daily data to each piece of health-related data in the second age group.

The prediction unit 45 has a function similar to that of the prediction unit 14 of the first example embodiment. The prediction unit further predicts the disease onset probability based on, for example, health-related data after a change has occurred due to daily life. The prediction unit 45 predicts the disease onset probability, for example, using a disease prediction model similar to that of the prediction unit 14 of the first example embodiment. For example, the prediction unit 45 predicts the disease onset probability using, as an input to the disease prediction model, health-related data in a case where the estimated age has increased based on health-related data after a change has occurred due to daily life. The prediction unit 45 may predict the disease onset probability using health-related data after a change has occurred due to daily life as an input to a disease prediction model. The prediction unit may predict the medical cost of the target person based on health-related data after a change has occurred due to daily life.

The output unit 46 has a function similar to that of the output unit of the first example embodiment. For example, in a case where the age of the target person has increased to the second age, the output unit 46 further outputs health-related data when the age has changed due to daily life. The output unit 46 may output, as health-related data in a case where the age of the target person has increased to the second age, health-related data when the age has changed due to daily life and health-related data that has not changed due to daily life. That is, the output unit 46 may output the estimation result of the health-related data in the second age group estimated from the estimated value of the health-related data in the first age group that has changed by daily life, and the estimation result of the health-related data in the second age group estimated from the measured value of the health-related data in the first age group.

The output unit 46 may output a prediction result of a medical cost of the target person in the future, which is estimated based on an estimation result of health-related data after a change has occurred due to daily life. The output unit 46 may output at least one of the health-related data of the target person in the future and the prediction result of the medical cost as the time series data. The time series data is, for example, a graph in which a horizontal axis is time and a vertical axis is health-related data or a prediction result of medical cost in a case where the age of the target person has increased.

The output unit 46 outputs, for example, an estimation result of health-related data to the terminal device 20. The output unit 46 may output the estimation result of the health-related data to the measurement device 50. The output unit 46 may output the estimation result of the health-related data to the terminal device 20 and the measurement device 50.

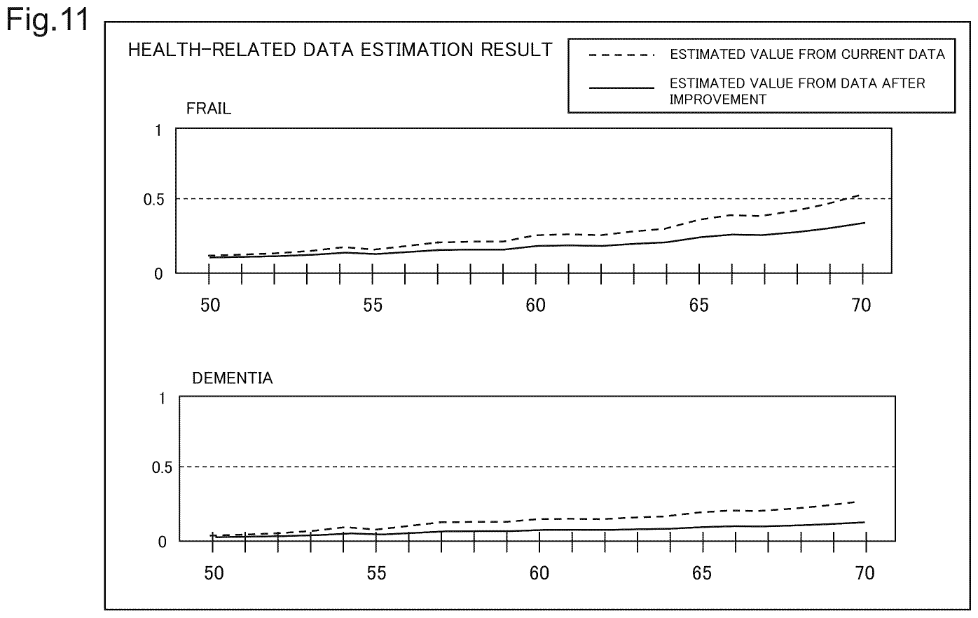

FIG. 11 is an example of a display screen that displays, as health-related data in a case where the age of the target person has increased, health-related data after a change has occurred due to daily life and health-related data that has not changed due to daily life as health-related data estimation results. In the example of the display screen of FIG. 11, a broken line indicates a temporal change of an estimated value of health-related data based on a current measured value. In the example of the display screen of FIG. 11, a solid line indicates a temporal change in an estimated value of health-related data based on an estimated value of health-related data after a change has occurred due to daily life.

In the example of the display screen in FIG. 11, the estimated value of the health-related data after a change has occurred due to daily life indicated by the solid line is displayed as “estimated value from data after improvement”. In the example of the display screen in FIG. 11, an estimated value of health-related data after a change has occurred due to daily life indicated by a solid line is displayed as an estimated value of health-related data in the future in a case where the current health-related data is improved as a result of behaviors in daily life, for example. For example, in a case where the target person who refers to the example of the display screen of FIG. 11 is walking in daily life, the target person can recognize the estimated value of the data improvement effect on health-related data caused by walking.

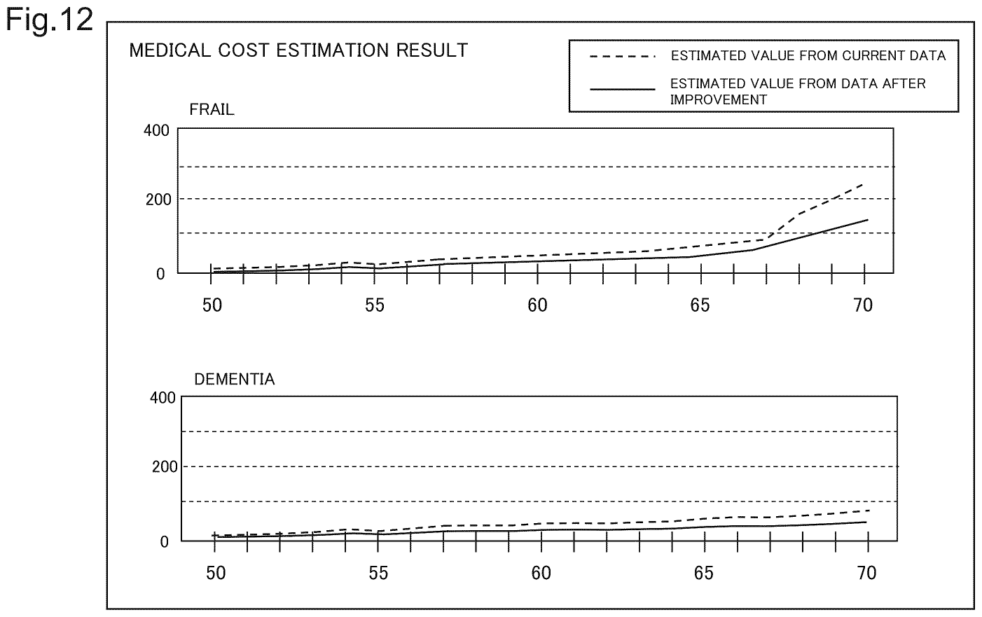

FIG. 12 is an example of a display screen that displays the cost for the target person to take medical care as health-related data in a case where the age of the target person has increased. In the example of the display screen of FIG. 12, a broken line indicates a temporal change in the estimated value of the future medical cost based on the current measured value. In the example of the display screen of FIG. 12, a solid line indicates a temporal change in an estimated value of future medical cost based on an estimated value of health-related data after a change has occurred due to daily life. In the example of the display screen in FIG. 12, the estimated value for taking medical care after a change has occurred due to daily life indicated by a solid line is displayed as the estimated value for taking medical care in the future in a case where health-related data is improved as a result of behavior in daily life, for example.

The storage unit 47 has a function similar to that of the storage unit 16 of the first example embodiment. The storage unit 47 further stores, for example, an estimation result of health-related data estimated based on daily data. The storage unit 47 stores the change estimation model. The change estimation model may be stored in storage means outside the data estimation device 40.

The measurement device 50 is, for example, a terminal device that acquires daily data of the target person. The measurement device 50 measures daily data of the target person using, for example, a sensor. Then, the measurement device 50 outputs the measured daily data to the acquisition unit 41 of the data estimation device 40. Some or all of the daily data of the target person may be input into the measurement device 50 by the target person. The daily data acquired by the measurement device 50 may be input to the data estimation device 40 using a removable storage medium. As the storage medium, for example, a nonvolatile semiconductor storage device can be used.

The measurement device 50 is not limited to a device dedicated to measurement. For example, the measurement device 50 may also include a multifunctional device such as a smartphone or a smart watch. The terminal device 20 and the measurement device 50 may be an integrated device.

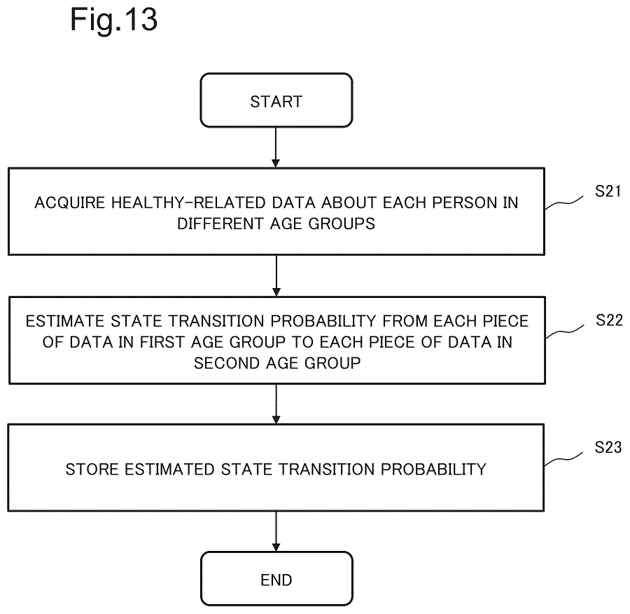

A process of calculating the state transition probability from each data group of the first age group to each data group of the second age group in the data estimation device 40 will be described. FIG. 13 illustrates an example of an operation flow in the process of calculating the state transition probability from each data group of the first age group to each data group of the second age group in the data estimation device 40.

For example, the acquisition unit 41 acquires health-related data of each person of different age groups (step S21). The acquisition unit 11 acquires health-related data from the data management device 30, for example.

When the health-related data is acquired, the estimation unit 44 calculates, for example, a state transition probability from each piece of data of the first age group to each piece of data of the second age group (step S22).

When the state transition probability is calculated, the estimation unit 44 stores, for example, the estimated state transition probability (step S23). For example, the estimation unit 44 stores the state transition probability for each combination of the predetermined item, the first age group, and the second age group in the storage unit 47 as a database.

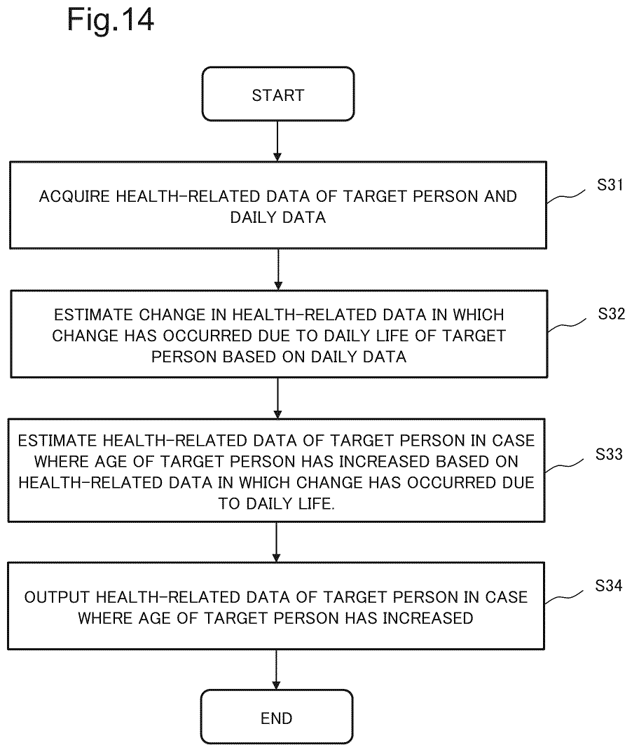

An operation in which the data estimation device 40 estimates health-related data in a case where the age of the target person has increased based on the health-related data in which a change has occurred due to daily life will be described. FIG. 14 is an example of an operation flow of processing of estimating health-related data in a case where the age of the target person has increased based on the health-related data in which a change has occurred due to daily life.

The acquisition unit 41 acquires, for example, health-related data of the target person and daily data (step S31). The acquisition unit 41 acquires, for example, daily data of the target person from the measurement device 50. The acquisition unit 41 acquires health-related data of the target person from the data management device 30, for example.

When the health-related data of the target person and the daily data are acquired, the change estimation unit 42 estimates, based on the daily data, for example, health-related data in which a change has occurred due to the daily life of the target person (step S32).

When the health-related data having changed due to daily life is estimated, the estimation unit 44 estimates the health-related data of the target person in a case where the age of the target person has increased based on the health-related data having changed due to daily life (step S33).

When the health-related data of the target person in a case where the age of the target person has increased is estimated, the output unit 46 outputs the health-related data of the target person in a case where the age of the target person has increased (step S34).

Each processing in the data estimation device 40 may be executed in a distributed manner in a plurality of information processing devices connected via a network. For example, the processing in the classification unit 43 and the estimation unit 44 and the processing in the change estimation unit 42 may be performed in another information processing device. Which information processing device performs each process in the data estimation device 40 can be appropriately set.

The data estimation device 40 estimates, for example, health-related data in which a change has occurred due to daily life of the target person based on the daily data. Then, the data estimation device 40 estimates the health-related data of the target person in a case where the age of the target person has increased based on the health-related data in which the change has occurred due to daily life. In this manner, the data estimation device 40 can estimate the future health condition that can be changed by the daily life by estimating the future health-related data based on the daily data in daily life. Therefore, the data estimation device 40 can improve the accuracy of the estimation of the future health condition of the target person.

For example, in a case where the health-related data is the result of the medical examination and the next medical examination is one year ahead, the data estimation device 40 can estimate the future health-related data of the target person based on the daily data, thereby estimating the health-related data reflecting the change in daily life, for example. Therefore, the target person can appropriately make a decision related to behavior improvement in daily life by referring to, for example, health-related data reflecting a change in daily life.

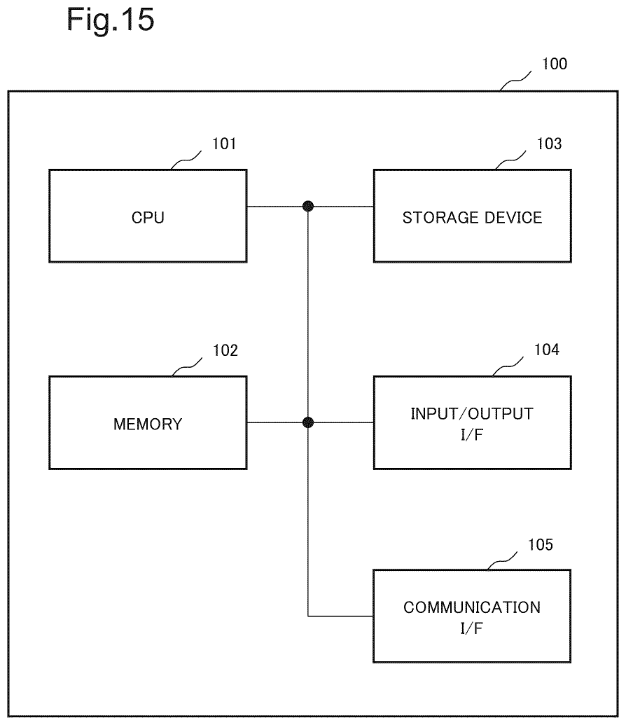

Each process in the data estimation device 10 and the data estimation device 40 can be implemented by executing a computer program on a computer. FIG. 15 illustrates an example of a configuration of a computer 100 that executes a computer program for executing each process in the data estimation device 10. The computer 100 includes a central processing unit (CPU) 101, a memory 102, a storage device 103, an input/output interface (I/F) 104, and a communication I/F105.

The CPU 101 reads and executes a computer program for executing each processing from the storage device 103. The CPU 101 may be configured by a combination of a plurality of CPUs. The CPU 101 may be configured by a combination of a CPU and another type of processor. For example, the CPU 101 may be configured by a combination of a CPU and a graphics processing unit (GPU). The memory 102 includes a dynamic random access memory (DRAM) or the like, and temporarily stores a computer program executed by the CPU 101 and data being processed. The storage device 103 stores a computer program executed by the CPU 101. The storage device 103 includes, for example, a nonvolatile semiconductor storage device. The storage device 103 may include another storage device such as a hard disk drive. The input/output I/F 104 is an interface that receives an input from an operator to output display data and the like. The communication I/F105 is an interface that transmits and receives data to and from the terminal device 20, the data management device 30, the measurement device 50, and other information processing devices. The terminal device 20 and the data management device 30 can also be configured as in the computer 100.

The computer program used for executing each processing can also be distributed by being stored in a computer-readable recording medium that non-transiently records data. The recording medium can include, for example, a magnetic tape for data recording or a magnetic disk such as a hard disk. The recording medium may include an optical disk such as a compact disc read only memory (CD-ROM). A non-volatile semiconductor storage device may be used as a recording medium.

The future health condition of a target person may be important in estimating assets necessary for receiving medical practice or the like in the future and grasping assets or the like that can be used for investment excluding assets necessary for receiving medical practice or the like. The estimation of the future health condition is performed, for example, based on the current health condition of the target person. For example, the estimation of the future health condition is performed using a machine learning model that predicts the future health condition from the health condition of the target person.

However, the technique described in JP 2016-173728 A requires health condition data for a plurality of years for the same person, and thus it may be difficult to estimate a future health condition.

In order to solve the above problem, an object of the present disclosure is to provide a data estimation device and the like that can easily estimate health-related data in the future.

According to the present disclosure, it is possible to easily estimate health-related data in the future.

Some or all of the above example embodiments may be described as the following Supplementary Notes, but are not limited to the following.

### [Supplementary Note 1]

A data estimation device including:
an acquisition unit that acquires health-related data of each person of different age groups;
an estimation unit that estimates a transition destination of health-related data of a target person in a case where an age of the target person in a first age group has become a second age group; and an output unit that outputs the health-related data in a case where the age of the target person has increased based on an estimation result of the transition destination.

### [Supplementary Note 2]

The data estimation device according to Supplementary Note 1, in which the estimation unit estimates a transition destination of health-related data of the target person using an algorithm related to an optimal transportation problem.

### [Supplementary Note 3]

The data estimation device according to Supplementary Note 1 or 2, further including a classification unit that classifies the health-related data into a data group in each age group, in which the estimation unit estimates a transition destination as a second data group in a case where transition is made from a first data group on a probability distribution of health-related data in the first age group to a data group on a probability distribution of health-related data in the second age group.

### [Supplementary Note 4]

The data estimation device according to Supplementary Note 2, in which the estimation unit s estimates health-related data in a case where the target person has increased to the second age group based on a state transition probability in a case where transition is made from the first data group on a probability distribution of health-related data in the first age group to a data group on a probability distribution of health-related data in the second age group.

### [Supplementary Note 5]

The data estimation device according to any one of Supplementary Notes 1 to 4, further including a prediction unit that predicts a disease onset probability in a case where an age of a person in the first age group has increased to the second age group based on health-related data in the second age group estimated as a transition destination of health-related data in the first age group.

### [Supplementary Note 6]

The data estimation device according to any one of Supplementary Notes 1 to 4, further including a change estimation unit that estimates health-related data after a change has occurred due to daily life of the target person based on daily data of the target person, in which the estimation unit estimates health-related data in a case where an age of the target person has increased to the second age group using the estimated health-related data as health-related data in the first age group.

### [Supplementary Note 7]

The data estimation device according to any one of Supplementary Notes 1 to 4, further including a prediction unit that predicts a disease onset probability in each of the health-related data for each of the age groups, in which the estimation unit estimates a transition destination of data of the disease onset probability of the target person in a case where an age of the target person becomes the second age group.

### [Supplementary Note 8]

The data estimation device according to any one of Supplementary Notes 1 to 7, in which the estimation unit estimates a transition destination of health-related data of a target person in a case where an age of the target person in the first age group becomes a second age group using an item related to a predetermined disease in the health-related data as a variable.

### [Supplementary Note 9]

The data estimation device according to Supplementary Note 5, in which the prediction unit predicts a disease onset probability in a case where an age of a person in the first age group has increased to the second age group using a machine learning model that predicts a disease onset probability based on health-related data.

### [Supplementary Note 10]

The data estimation device according to any one of Supplementary Notes 1 to 9, in which the estimation unit estimates a transition destination of health-related data of a target person in a case where an age of the target person in the first age group becomes a second age group via the health-related data in at least one or more age groups between the first age group and the second age group.

### [Supplementary Note 11]

The data estimation device according to any one of Supplementary Notes 1 to 10, in which the output unit outputs health-related data of the target person in the first age group and health-related data in a case where an age of the target person increases to the second age group.

### [Supplementary Note 12]

The data estimation device according to Supplementary Note 6, in which the output unit outputs health-related data when an age of the target person has changed in daily life and health-related data that has not changed in daily life as health-related data when an age of the target person has increased to the second age group.

### [Supplementary Note 13]

The data estimation device according to Supplementary Note 7, in which the output unit outputs, as the health-related data, a disease onset probability in a case where an age of a target person in the first age group becomes the second age group.

### [Supplementary Note 14]

The data estimation device according to any one of Supplementary Notes 1 to 13, in which the acquisition unit acquires a result of a medical examination within a predetermined period and/or in a predetermined group as the health-related data.

### [Supplementary Note 15]

The data estimation device according to any one of Supplementary Notes 1 to 14, in which the classification unit classifies, into the data group, the health-related data of a person having a same attribute as that of the target person among the acquired health-related data.

### [Supplementary Note 16]

A data estimation method including:
acquiring health-related data of each person of different age groups;
estimating a transition destination of health-related data of a target person in a case where an age of the target person in a first age group has become a second age group; and
outputting the health-related data in a case where the age of the target person has increased based on an estimation result of the transition destination.

### [Supplementary Note 17]

A non-transitory recording medium recording a data estimation program for causing a computer to execute:
a process of acquiring health-related data of each person of different age groups;
a process of estimating a transition destination of health-related data of a target person in a case where an age of the target person in a first age group has become a second age group; and
a process of outputting the health-related data in a case where the age of the target person has increased based on an estimation result of the transition destination.

Some or all of the configurations described in Supplementary Notes 2 to 15 dependent on the above-described Supplementary Note 1 can also be dependent on Supplementary Notes 16 and 17 by the same dependency relationship as in Supplementary Notes 2 to 15. Furthermore, some or all of the configurations described as the Supplementary Notes can be similarly dependent on not only the Supplementary Notes 1, 16, and 17, but also various pieces of hardware and software, and various recording means or systems for recording software without departing from the above-described example embodiments.

The previous description of embodiments is provided to enable a person skilled in the art to make and use the present disclosure. Moreover, various modifications to these example embodiments will be readily apparent to those skilled in the art, and the generic principles and specific examples defined herein may be applied to other embodiments without the use of inventive faculty. Therefore, the present disclosure is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

1. A data estimation device comprising:
at least one memory storing instructions; and
at least one processor configured to access the at least one memory and execute the instructions to:
acquire health-related data of each person of different age groups;
estimate a transition destination of health-related data of a target person in a case where an age of the target person in a first age group has become a second age group; and
output the health-related data in a case where the age of the target person has increased based on an estimation result of the transition destination.

2. The data estimation device according to claim 1, wherein
the at least one processor is further configured to execute the instructions to:
estimate a transition destination of health-related data of the target person using an algorithm related to an optimal transportation problem.

3. The data estimation device according to claim 2, wherein
the at least one processor is further configured to execute the instructions to:
classify the health-related data into a data group in each age group; and
estimate a transition destination as a second data group in a case where transition is made from a first data group on a probability distribution of health-related data in the first age group to a data group on a probability distribution of health-related data in the second age group.

4. The data estimation device according to claim 3, wherein
the at least one processor is further configured to execute the instructions to:
estimate health-related data in a case where the target person has increased to the second age group based on a state transition probability in a case where transition is made from the first data group on a probability distribution of health-related data in the first age group to a data group on a probability distribution of health-related data in the second age group.

5. The data estimation device according to claim 1, wherein
the at least one processor is further configured to execute the instructions to:
predict a disease onset probability in a case where an age of a person in the first age group has increased to the second age group based on health-related data in the second age group estimated as a transition destination of health-related data in the first age group.

6. The data estimation device according to claim 1, wherein
the at least one processor is further configured to execute the instructions to:
estimate health-related data after a change has occurred due to daily life of the target person based on daily data of the target person; and
estimate health-related data in a case where an age of the target person has increased to the second age group using the estimated health-related data as health-related data in the first age group.

7. The data estimation device according to claim 1, wherein the at least one processor is further configured to execute the instructions to:
predict a disease onset probability in each of the health-related data for each of the age groups; and
estimate a transition destination of data of the disease onset probability of the target person in a case where an age of the target person becomes the second age group.

8. The data estimation device according to claim 1, wherein
the at least one processor is further configured to execute the instructions to:
estimate a transition destination of health-related data of a target person in a case where an age of the target person in the first age group becomes a second age group using an item related to a predetermined disease in the health-related data as a variable.

9. The data estimation device according to claim 5, wherein
the at least one processor is further configured to execute the instructions to:
predict a disease onset probability in a case where an age of a person in the first age group has increased to the second age group using a machine learning model that predicts a disease onset probability based on health-related data.

10. The data estimation device according to claim 1, wherein
the at least one processor is further configured to execute the instructions to:
estimate a transition destination of health-related data of a target person in a case where an age of the target person in the first age group becomes a second age group via the health-related data in at least one or more age groups between the first age group and the second age group.

11. The data estimation device according to claim 1, wherein
the at least one processor is further configured to execute the instructions to:
output health-related data of the target person in the first age group and health-related data in a case where an age of the target person increases to the second age group.

12. The data estimation device according to claim 1, wherein
the at least one processor is further configured to execute the instructions to:
output health-related data when an age of the target person has changed in daily life and health-related data that has not changed in daily life as health-related data when an age of the target person has increased to the second age group.

13. The data estimation device according to claim 7, wherein
the at least one processor is further configured to execute the instructions to:
output, as the health-related data, a disease onset probability in a case where an age of a target person in the first age group becomes the second age group.

14. The data estimation device according to claim 1, wherein
the at least one processor is further configured to execute the instructions to:
acquire a result of a medical examination within a predetermined period and/or in a predetermined group as the health-related data.

15. The data estimation device according to claim 1, wherein
the at least one processor is further configured to execute the instructions to:
classify, into the data group, the health-related data of a person having a same attribute as that of the target person among the acquired health-related data.

16. A data estimation method comprising:
acquiring health-related data of each person of different age groups;
estimating a transition destination of health-related data of a target person in a case where an age of the target person in a first age group has become a second age group; and
outputting the health-related data in a case where the age of the target person has increased based on an estimation result of the transition destination.

17. A non-transitory recording medium recording a data estimation program for causing a computer to execute:
a process of acquiring health-related data of each person of different age groups;
a process of estimating a transition destination of health-related data of a target person in a case where an age of the target person in a first age group has become a second age group; and
a process of outputting the health-related data in a case where the age of the target person has increased based on an estimation result of the transition destination.
